(19) United States
(12) Patent Application Publication
(10) Pub. No.: US 2012/0134973 A1
(43) Pub. Date: May 31, 2012

(54) NOVEL USE OF PROBIOTICS

(75) Inventor: Riina Kekkonen, Helsinki (FI)

(73) Assignee: VALIO LTD., Helsinki (FI)

(21) Appl. No.: 13/319,458

(22) PCT Filed: May 12, 2010
(86) PCT No.: PCT/EP2010/056553

§ 371 Date: Jan. 18, 2012

(30) Foreign Application Priority Data
May 12, 2009 (FI) 20095529

(51) Int. Cl. A61K 35/74; A61K 36/06; A61P 9/00; A61P 3/00; A61P 3/04; A61K 35/66; A61P 29/00

(52) U.S. Cl. 424/93.44; 424/93.1; 424/93.45; 424/93.51

(57) ABSTRACT

The present invention relates to use of a probiotic to normalize abnormal inflammation markers. The present invention also relates to use of a probiotic for preventing and/or treating low-grade inflammation. Further, the present invention relates to use of a probiotic for preventing and/or treating disorders and/or diseases relating to low-grade inflammation.

### FIELD OF THE INVENTION

The present invention relates to use of probiotics to normalize abnormal inflammation markers. The present invention also relates to use of a probiotic for preventing and/or treating low-grade inflammation. Further, the present invention relates to use of probiotics for preventing and/or treating disorders and/or diseases relating to low-grade inflammation.

### BACKGROUND OF THE INVENTION

Inflammation is a central mechanism contributing to the progression of cardiovascular diseases. Cardiovascular risk factors and metabolic syndrome are typified by low-grade inflammation, with recent evidence pointing toward the presence of an important component dependent on a low-grade inflammatory process. Inflammatory markers (e.g. C-reactive protein CRP, chemokines and adhesion molecules) are increased in patients with hypertension and metabolic disorders, and predict the development of cardiovascular disease.

Savoia and Schiffrin (Clin Sci (Lond), 2007 June; 112 (7): 375-84) have reviewed vascular inflammation in hypertension and diabetes with a special focus on the pathophysiological role of low-grade inflammation in the vasculature of patients with hypertension and diabetes, as well as the role of inflammatory markers in cardiovascular disease, in view of potential therapeutic interventions to reduce cardiovascular risk. Patients with cardiovascular disease present with increased expression and plasma concentration of inflammatory markers and mediators, which include CRP and adhesion molecules, such as selectins (P-selectin, E-selectin and L-selectin), ICAM-1 (intercellular adhesion molecule-1) and VCAM-1 (vascular cell adhesion molecule-1). Moreover, increased plasma levels of the primary inflammatory cytokine TNF-α (tumour necrosis factor-α), and the secondary inflammatory cytokine IL-6 (interleukin-6), as well as ICAM-1, VCAM-1, E-selectin, vWF (von Willebrand factor) and CRP, have been demonstrated in patients with hypertension. High levels of inflammatory mediators, particularly IL-6, ICAM-1 and CRP, may be independent risk factors for the development of hypertension, increased risk of diabetes and cardiovascular disease. Inflammation measured by these markers, mainly CRP, may be included in the definition of the metabolic syndrome, a constellation of abnormalities (including abdominal obesity, high blood glucose/impaired glucose tolerance, dyslipidaemia and high blood pressure) that increase the risk of overt diabetes mellitus and cardiovascular events. Lowering blood pressure as well as therapeutic approaches to control vascular inflammation, particularly in patients with glucose intolerance or diabetes, may provide significant clinical benefits.

Fulop T et al., (Pathol Biol (Paris), 2006 September; 54(7): 375-86) discloses that the metabolic syndrome (MS) is a cluster of metabolic abnormalities leading to increased risk for cardiovascular diseases and diabetes type 2. Visceral obesity and the resulting insulin resistance are the major determinant in the development of the MS. Abdominal obesity results in a low-grade inflammation via the adipose tissue and macrophages secreted adipokines. This inflammation, via the generated pro-inflammatory molecules, interferes with the normal insulin signalling and thus contributes to the etiopathogenesis of the MS. CRP is increased in obese subjects and concomitantly to the number of existing component of the MS. Treatment of the MS is aimed to improve the insulin resistance by lifestyle changes including exercise and diet alone or in combination with medication targeting the individual components but having also anti-inflammatory actions.

Currently, lifestyle modifications such as weight loss, physical exercise, diet modifications in macro- and micro-nutrients and/or Mediterranean-style diet and pharmacological approaches (such as drugs with specific target i.e. rennin-angiotensin system) are used in the treatment and prevention MS. Lifestyle modification and pharmacological approaches may reduce blood pressure and inflammation in patients with hypertension and metabolic disorders, which will reduce cardiovascular risk, development of diabetes and cardiovascular morbidity and mortality. However, longterm benefits of moderate weight loss by lifestyle modifications are limited. Furthermore, lifestyle changes do not seem to be sufficient alone. Medication, i.e. insulin sensitisers (glitazones, mefformines, thiazolidionediones), lipid modifiers (statins, fibrates), inhibitors of angiotensine converting enzyme and angiotensine receptor antagonists has been studied.

According to Basu et al. (Aterioscler Thromb Vasc Biol. 2006 May; 26(5): 995-1001) intervention trials convincingly demonstrate that weight loss reduces biomarkers of inflammation, such as CRP and IL-6. Limited studies have shown that certain dietary factors; oleic acid, alpha-linolenic acid, and antioxidants RRR-alpha-alpha tocopherol, reduce biomarkers of inflammation.

Publication WO 2007/043933 discloses use of probiotic bacteria being selected from Lactobacillus casei F19 (LGM P-17806), Lactobacillus acidophilus NCFB 1748 and Bifidobacterium lactis Bb12 for controlling weight gain, preventing obesity, increasing satiety, prolonging satiation, reducing food intake, reducing fat deposition, improving energy metabolism, enhancing insulin sensitivity, treating obesity and treating insensitivity.

Kekkonen et al. (World J. Gastroenterol. 2008; 14, 2029-2036) discreibed that in a three-month intervention study in healthy adults, serum highly sensitive CRP levels were reduced in the Lactobacillus rhamnosus GG (LGG) and Propionibacterium freudenreichii ssp. shermanii JS (PJS) groups. Further, according to Kekkonen et al. (publication IV in Kekkonen's doctoral thesis of Jun. 6, 2008) consumption of LGG resulted in decreased serum CRP levels as compared to the controls within exercising adults participating in marathon.

Novel therapeutic approaches are needed to decrease the incidence and prevalence of low-grade inflammation, especially diet-induced low-grade inflammation among population. Novel therapeutic approaches are needed also to decrease the incidence the metabolic syndrome (MS) among the population.

The present invention now provides a novel indication of probiotics.

### BRIEF DESCRIPTION OF THE INVENTION

The present invention relates to use of a probiotic for preventing, alleviating and/or treating low-grade inflammation. Specifically, the present invention relates to the use of a probiotic for preventing, alleviating and/or treating low-grade inflammation induced by a diet. Thus, the present invention relates also to a probiotic for use in alleviating, preventing and/or treating diet-induced low-grade inflammation. In one embodiment, the invention relates to the use of a probiotic for preventing, alleviating and/or treating low-grade inflammation induced by fats in the diet. In another embodiment, the invention relates to the use of a probiotic for preventing, alleviating and/or treating low-grade inflammation induced by a high-fat diet or a hyperlipidemic diet.

The present invention also relates to use of a probiotic for preventing and/or treating disorders and/or diseases relating to and/or associated with low-grade inflammation. Specifically, the present invention relates to the use of a probiotic for preventing and/or treating diseases relating to and/or associated with a diet-induced low-grade inflammation. Thus, the present invention also relates to a probiotic for use in preventing and/or treating diseases relating to and/or associated with a diet-induced low-grade inflammation. The present invention further relates to use of a probiotic to suppress inflammation markers and/or to normalize abnormal inflammation markers. Specifically, the invention relates to use of a probiotic for suppressing diet-induced inflammation markers, and/or for normalizing abnormal diet-induced inflammation markers, such as markers formed in liver, adipose tissue and/or vasculature, as well as alleviating, preventing and/or treating disorders and diseases relating thereto. Thus, the present invention relates also to a probiotic for use in normalizing an abnormal diet-induced low-grade inflammation marker as well as for use in suppressing a diet-induced low-grade inflammation marker. In one embodiment, the present invention relates to use of a probiotic for suppressing inflammation markers induced by fats in the diet, and/or for normalizing abnormal inflammation markers inflammation induced by fats in the diet, as well as alleviating, preventing and/or treating disorders and diseases relating thereto. In another embodiment, the present invention relates to use of a probiotic for suppressing high-fat diet-induced inflammation markers, and/or for normalizing abnormal, high-fat diet-induced, inflammation markers, as well as alleviating, preventing and/or treating disorders and diseases relating thereto. In one embodiment of the invention the probiotic is selected from L. rhamnosus GG (LGG) (ATCC 53103), L. rhamnosus LC705 (DSM 7061), and/or P. freudenreichii ssp. shermanii JS (DSM 7067).

The invention also relates to novel use of a probiotic, especially a probiotic strain L. rhamnosus GG (LGG) (ATCC 53103), L. rhamnosus LC705 (DSM 7061), and/or P. freudenreichii ssp. shermanii JS (DSM 7067) or a mixture thereof for decreasing the risk of developing metabolic syndrome, obesity, especially abdominal obesity, cardiovascular diseases and/or diabetes type 2, or preventing and/or treating metabolic syndrome, obesity, especially abdominal obesity, cardiovascular diseases and/or diabetes type 2. In addition, the invention also relates to a use of a probiotic, especially a probiotic strain L. rhamnosus GG (LGG) (ATCC 53103), L. rhamnosus LC705 (DSM 7061), and/or P. freudenreichii ssp. shermanii JS (DSM 7067) or a mixture thereof in weight control of an individual. Accordingly, the present invention relates also to a probiotic, especially a probiotic strain L. rhamnosus GG (LGG) (ATCC 53103), L. rhamnosus LC705 (DSM 7061), and/or P. freudenreichii ssp. shermanii JS (DSM 7067) or a mixture thereof, for use in decreasing the risk of developing metabolic syndrome, obesity, especially abdominal obesity, cardiovascular diseases and/or diabetes type 2, or preventing and/or treating metabolic syndrome, obesity, especially abdominal obesity, cardiovascular diseases and/or diabetes type 2.

The present invention also relates to a method for preventing, alleviating and/or treating low-grade inflammation by administering to an individual a probiotic in a sufficient amount to produce the desired effect. Specifically, the present invention relates to the method for preventing, alleviating and/or treating low-grade inflammation induced by a diet. In one embodiment, the invention relates to the method for preventing and/or treating low-grade inflammation induced by fats in the diet. In another embodiment, the invention relates to the method for preventing and/or treating low-grade inflammation induced by a high-fat diet or a hyperlipidemic diet. The present invention also relates to a method for preventing and/or treating disorders and/or diseases relating to and/or associated with low-grade inflammation by administering to an individual a probiotic in a sufficient amount to produce the desired effect. The present invention further relates to a method for suppressing inflammation markers and/or for normalizing abnormal inflammation markers. Specifically, the invention relates to a method for suppressing diet-induced inflammation markers, and/or normalizing abnormal diet-induced inflammation markers, especially markers formed in liver, adipose tissue and/or vasculature, as well as alleviating, preventing and/or treating disorders and diseases relating thereto, by administering to an individual a probiotic in a sufficient amount to produce the desired effect. In one embodiment, the present invention relates to a method for suppressing inflammation markers induced by fats in the diet, and/or normalizing abnormal inflammation markers induces by fats in the diet, as well as alleviating, preventing and/or treating disorders and diseases relating thereto, by administering to an individual a probiotic in a sufficient amount to produce the desired effect. In another embodiment, the present invention relates to a method for suppressing high-fat and/or hyperlipidemic diet-induced inflammation markers, and/or normalizing abnormal high-fat and/or hyperlipidemic diet-induced inflammation markers, as well as alleviating, preventing and/or treating disorders and diseases relating thereto, by administering to an individual a probiotic in a sufficient amount to produce the desired effect. In a further embodiment of the invention the probiotic is selected from L. rhamnosus GG (LGG) (ATCC 53103), L. rhamnosus LC705 (DSM 7061), and/or P. freudenreichii ssp. shermanii JS (DSM 7067) or a mixture thereof.

The present invention further relates to a method for decreasing the risk of developing metabolic syndrome, obesity, especially abdominal obesity, cardiovascular diseases and/or diabetes type 2, or for preventing and/or treating metabolic syndrome, obesity, especially abdominal obesity, cardiovascular diseases and/or diabetes type 2, by administering to a subject a probiotic, especially a probiotic strain Lactobacillus rhamnosus GG (LGG), L. rhamnosus LC705 and/or Propionibacterium freudenreichii ssp. shermanii JS or a mixture thereof. In addition, the invention also relates to a method for controlling weight of an individual by administering a probiotic, especially a probiotic strain L. rhamnosus GG (LGG) (ATCC 53103), L. rhamnosus LC705 (DSM 7061), and/or P. freudenreichii ssp. shermanii JS (DSM 7067) or a mixture thereof to the subject or individual.

The objects of the invention are achieved by the products, methods and uses set forth in the independent claims. Preferred embodiments of the invention are described in the dependent claims.

Other objects, details and advantages of the present invention will become apparent from the following drawings, detailed description and examples.

### BRIEF DESCRIPTION OF THE DRAWINGS

FIG. 1 shows the effect of a probiotic on alanine amino transferase (ALAT). Values are mean values at group level.

GG=Lactobacillus rhamnosus GG (LGG), JS=Propionibacterium freudenreichii ssp. shermanii JS, FF=positive control group (0.003% fenofibrate).

FIG. 2 A-D shows the effect of a probiotic on plasma SAA levels and relative (corrected) SAA levels for Water-consuming groups (A; C) and milk-consuming groups (B; D). Values are absolute mean values. To correct for the differences in SAA at t=0, effects on SAA were also analyzed relative to the t=0 value, i.e. the t=0 value was set 100% for each group. Values are mean±SD.

GG=Lactobacillus rhamnosus GG (LGG), JS=Propionibacterium freudenreichii ssp. shermanii JS, FF=positive control group (0.003% fenofibrate).

FIG. 3 A-B shows the effect of a probiotic on plasma sE-selectin concentrations for Water-consuming groups (A) and milk-consuming groups (B). Values are absolute means±SD values.

GG=Lactobacillus rhamnosus GG (LGG), JS=Propionibacterium freudenreichii ssp. shermanii JS, FF=positive control group (0.003% fenofibrate).

FIG. 4 A-B shows the effect of a probiotic on plasma sVCAM-1 (Vascular Cell Adhesion Molecule) concentrations for Water-consuming groups (A) and milk-consuming groups (B). Values are absolute means±SD values.

GG=Lactobacillus rhamnosus GG (LGG), JS=Propionibacterium freudenreichii ssp. shermanii JS, FF=positive control group (0.003% fenofibrate).

### DETAILED DESCRIPTION OF THE INVENTION

Metabolic syndrome and disorders and diseases relating to metabolic syndrome, such as obesity, particularly abdominal obesity, cardiovascular diseases and diabetes type 2 are common diseases among the population, especially in industrialized countries nowadays. As is true with other medical conditions, in addition to genetics, the environment plays important role in the development of the metabolic syndrome. Environmental issues such as exiguity of physical exercise, sedentary lifestyle, and progressive weight gain, especially an increase in body fat as a result of a diet, contribute significantly to the risk of developing the metabolic syndrome. Lifestyle modification is the preferred treatment of metabolic syndrome. Weight reduction usually requires a specifically tailored diet as well as exercise.

Low-grade inflammation occurs typically in vasculature and adipose tissue of a subject. Low-grade inflammation is typically chronic in its nature. In the present invention, the term “low-grade inflammation” refers to an inflammatory state wherein the C-reactive protein (CRP) is less than 10.0 mg/l, specifically from 3 to 10 mg/l. CRP, especially high-sensitivity CRP (hs-CRP) analysis are done today using immunological methods Several factors, such as different diseases or disorders, are known to induce or to be associated with low-grade inflammation. One of the factors associated with low-grade inflammation is the diet and/or the nutritive ingredients, such as fats, and their relative amounts in the diet. A high-fat and/or a hyperlipidemic diet induces disorders in lipid metabolism of an individual. A high-fat diet or a hyperlipidemic diet contains nutritional components, specifically fatty substances such as saturated fatty acids that irritate the system of an individual and cause low-grade inflammation. High-fat diet and/or hyperlipidemic diet refers generally to a diet having a nutritional composition of which at least 30% of the total energy originates from fats. The terms “high-fat diet” and “hyperlipidemic diet” are typically also used to refer to a diet, the fatty acid composition of which being non-optimal, i.e., more than ⅓ of the fatty acids being saturated fatty acids. Thus, in addition to the total fat content of the diet, the quality of the fat in the diet, such as the ratio of unsaturated fatty acids to saturated fatty acids, is an important factor in determining a diet as a high-fat and/or hyperlipidemic diet.

Now, it has been found that a probiotic is able to prevent and/or treat low-grade inflammation, especially when the low-grade inflammation is induced by a diet, especially by fats in the diet and particularly by a high-fat diet or a hyperlipidemic diet. This is an important finding that could be used in preventing, alleviating and/or treating in addition to low-grade inflammation also disorders and/or diseases related to and/or associated with low-grade inflammation. This finding could also be used in developing means for preventing and/or treating disorders and/or diseases related to and/or associated with low-grade inflammation, especially diet-induced low-grade inflammation.

The present invention resides in the surprising finding that a probiotic is capable of suppressing diet-induced inflammation markers formed in liver and in vasculature and/or normalizing abnormal levels of the markers. An ability of a probiotic to suppress diet-induced markers of low-grade inflammation formed in the system is an especially important feature of the present invention. In an animal experiment with mice, it was found that a probiotic was able to suppress an inflammatory response at 3 days after high fat diet feeding. The probiotic quenched this acute inflammatory effect arising from high-fat diet feeding both with respect to hepatic and vascular inflammatory markers. The probiotic was found to reduce the inflammation markers in the long run, i.e. to levels lower than the control.

Furthermore, in the presence of milk-based material, the effect and/or the potency of a probiotic was found to be higher than in combination with water. In the animal test with mice, in the milk-consuming group, the acute anti-inflammatory effects of a probiotic were more pronounced than with water-consuming group.

The invention thus provides a novel use of a probiotic for preventing, alleviating and/or treating low-grade inflammation, especially diet-induced low-grade inflammation. Particularly, the invention provides a novel use of a probiotic for preventing alleviating and/or treating low-grade inflammation induced by fats in a diet. Further, the invention provides a novel use of a probiotic for preventing, alleviating and/or treating low-grade inflammation induced by and/or during a high-fat and/or hyperlipidemic diet.

The present invention is directed to novel use of probiotic(s) that as such or as a part of regular diet, or as a food supplement, or a medical or pharmaceutical product is capable of preventing, alleviating, treating or curing low-grade inflammation as well as disorders and/or diseases relating or associated thereto, such as metabolic syndrome, obesity, cardiovascular diseases and/or diabetes type 2. The present invention is also directed to novel use of probiotic(s) as such, or as a part of regular diet, or as a food supplement, or a medical or pharmaceutical product in weight control of an individual.

Especially, present invention is directed to use of L. rhamnosus GG (LGG) (ATCC 53103), L. rhamnosus LC705 (DSM 7061), and/or P. freudenreichii ssp. shermanii JS (DSM 7067) or a mixture thereof that as part of regular diet, or as a food supplement, or a medical or pharmaceutical product is capable of preventing, alleviating, treating or curing low-grade inflammation as well as disorders and/or diseases relating or associated thereto, such as metabolic syndrome, obesity, cardiovascular diseases and/or diabetes type 2.

The present invention relates also to a method for preventing, alleviating or treating low-grade inflammation as well as disorders and/or diseases relating thereto by administering to an individual a probiotic or an edible product containing a probiotic, in a sufficient amount to produce the desired effect in the individual. Especially, the present invention relates to a method for preventing, alleviating or treating low-grade inflammation during high-fat diet or hyperlipidemic diet, as well as disorders and diseases relating thereto, by administering to an individual a probiotic/probiotics or a product containing the probiotic(s).

The present invention also relates to a use of a probiotic for suppressing markers and/or normalizing abnormal markers of low-grade inflammation formed in the system, especially in liver, adipose tissue and/or vasculature, and a method for suppressing and/or normalizing abnormal markers of low-grade inflammation formed in the system, especially in liver, adipose tissue and/or vasculature by administering to an individual subject a probiotic or an edible product containing the probiotic, in a sufficient amount to produce the desired effect in the individual. In one embodiment of the invention, the markers of low-grade inflammation formed in the individual's system are diet-induced, particularly induced by fats in his diet. In another embodiment of the invention, the markers of low-grade inflammation formed in the system are high-fat and/or hyperlipidemic diet-induced.

A microorganism may be referred to as a “probiotic”, if it essentially meets the following requirements: it remains viable in the demanding conditions prevailing in the digestive tract (low pH of the stomach, acids of the digestive system, etc.); attaches to the walls of the intestine; metabolizes in the intestine; is technologically applicable (endures processing); exhibits clinically tested and reported health effects; and is safe to consume (Lee, Y-K and Salminen, S, Trends Food Sci Technol, 1995, 6: 241-245). The best-known probiotics are bacteria, especially lactic acid bacteria. The probiotic(s) to be used in the invention are preferably selected from the group consisting of lactobacilli, propionibacteria, bifidobacteria, lactococci, enterococci, streptococci, yeast and any combinations thereof. Preferably, the probiotic belongs to the genera Lactobacillus, preferably to the species Lactobacillus rhamnosus, and most preferably L. rhamnosus GG (LGG) or L. rhamnosus LC705 (LC705). In one embodiment of the invention, the probiotic is P. freudenreichii ssp. shermanii JS (DSM 7067).

The probiotic is conveniently administered as an oral composition containing metabolically active, i.e., live and/or lyophilized, or non-viable heat-killed, irradiated or lysed probiotic microorganisms.

The probiotic can be administered orally as such, i.e., in the form of a tablet, capsule or powder. In addition, the probiotic can be administered orally as a food or nutritional product, such as a milk or whey based fermented dairy product, or as a food supplement or a pharmaceutical product. According to one embodiment of the invention the product is an edible product, such as a dairy product, drink, juice, soup or children's food.

The probiotic may optionally be combined with at least one suitable prebiotic compound. A “prebiotic” is usually a non-digestible carbohydrate such as an oligo- or polysaccharide, or a sugar alcohol, which is not degraded or absorbed in the upper digestive tract. Known commercially used prebiotics include inulin, fructo-oligosaccharides, oligofructose or galacto-oligosaccharides.

The term “edible product” is intended to cover all consumable products, especially food products, and it can be solid, jellied or liquid. The term covers both ready-made products and products, which are produced by using the probiotic composition as a starter alone, or in combination with conventional starters or other probiotics. The food products can for instance be products of the dairy industry or beverage industry. Alternatively it can be a natural product.

In the present invention, “dairy product” means any liquid or semi-solid milk or whey based product having a varying fat content. The dairy product can be, e.g., cow's milk, goat's milk, sheep's milk, cream, full-fat milk, whole milk, low-fat milk or skim milk, ultrafiltered milk, diafiltered milk, microfiltered milk, or recombined milk from powdered milk and whey without any processing, or a processed product, such as yoghurt, curdled milk, curd, sour milk, sour whole milk, butter milk, other sour milk products, such as viili, filling of snack bars, etc. Another important group includes milk beverages, such as whey beverages, fermented milks, condensed milks, infant or baby milks; icecream; milk-containing food such as sweets.

In one embodiment of the invention, the probiotic is formulated into a milk-based product or a fermented dairy product or it is used in the preparation of a milk-based product or a fermented dairy product. The probiotic and the starter, if any, are used in a balanced proportion to each other in the production. The selection of suitable methods and preparation conditions belongs to knowledge of a person skilled in the art.

The dairy or milk-based products described above can be used as such to achieve the desired effect. Said products can also be concentrated and used as ingredients. Further, the products can also be dried and used in the form of powder or lyophilisate. The products are also applicable as capsules, pills or tablets, for example, manufactured in conventional processes used in the preparation of such product for example in the pharmaceutical industry. The products can also be used in the preparation of functional food products, health and wellness promoting edible products, or other corresponding ingredients, products or supplements. It may also be an animal feed. Thus, the form of each of the food or feed product, food supplement or ingredient, and/or the pharmaceutical product is not particularly limited. The probiotic can be formulated into an edible or enterally or orally administered product.

The probiotic and the products described herein are primarily suitable for use for human adults and infants. The positive effects of the products are also beneficial to animals, especially pets and production animals. Examples of these include dogs, cats, rabbits, horses, cows, pigs, goats, sheep and poultry. The term “subject” and the term “individual” as used herein thus includes both humans and animals.

In one embodiment of the invention, the probiotic is formulated into a functional food product comprising at least one probiotic that as part of a regular diet prevents or treats low-grade inflammation and/or disorders and/or diseases relating to low-grade inflammation.

In another further embodiment of the invention the probiotic composition of the invention is a food ingredient or food supplement comprising at least one probiotic that prevents or treats low-grade inflammation and/or disorders and/or diseases relating to low-grade inflammation.

In yet another embodiment of the present invention, the probiotic is formulated into a medical or a pharmaceutical product comprising at least one probiotic that prevents or treats low-grade inflammation and/or disorders and/or diseases relating to low-grade inflammation.

The probiotics are administered in an amount sufficient to prevent or treat low-grade inflammation and/or diseases and/or disorders relating to low-grade inflammation in a subject. Biologically effective amounts of probiotics have been previously described. The levels of hsCRP and/or diet-induced inflammation markers formed in liver (such as SAA) and vasculature (such as E-selectin, VCAM-1) of a subject suffering from low-grade inflammation differ from ones of healthy controls. An effective amount of the probiotic is an amount that is able to normalize by up- or down-regulation the increased or decreased level of at least one of the abnormal inflammation markers. An effective daily dose of a probiotic is typically from about 10⁶ to about 10¹⁰ cfu.

The probiotic(s) and/or the probiotic composition of the invention can be added to a product during its preparation or to a finished product. The food, feed and/or pharmaceutical products in question thus contain the desired characteristics on diet-induced inflammation markers formed in the system of a subject.

The invention will be described in more detail by means of the following examples. The examples are not to be construed to limit the claims in any manner whatsoever.

### Example 1

Anti-inflammatory and plasma lipid-modulating potency of probiotic bacterial strains (Lactobacillus rhamnosus GG and Propionibacterium freudenreichii ssp. shermanii JS) alone and in combination with milk, using male APOE*3Leiden transgenic mice fed a high fat diet were analyzed.

Mice. Male heterozygous APOE*3Leiden (E3L) mice were housed during the experiment in clean-conventional humidity and temperature-controlled animal rooms (relative humidity 50-60%, temperature ˜21° C., a 12-h light/dark cycle). Mice were supplied with food and acidified tap water ad lib or milk prepared from a fat-free low-lactose milk powder (Valio Ltd, Finland; protein 3.5%, sugars 5.2% of which lactose 1.0%, fat<1.0% of which saturated fatty acids 0.7%, sodium 0.42%, calcium 1200 mg/100 g) and acidified tap water ad lib. Mice were housed in macrolon cages (six mice or less per cage). The age of the mice at the beginning of the experiment was 15 to 17 weeks.

Animal welfare. Experiments were performed in accordance with the rules and regulations of the Netherlands Law on Animal Experiments, and the institutional ethics Committee on Animal Experiments (DEC) approved the protocol.

Diets. A high fat diet powder (Van den Hoek A M, et al., Diabetes, 2004; 53:1949-1952) provided by Hope Farms (Woerden, the Netherlands; crude protein 21.4%, crude fiber 6.16%, crude fat 24.0%, minerals 2.25%, calcium 863 mg/100 g, moisture 5.57%) was used. Pellets were prepared by mixing the powdered diet with 2% agar and freeze-drying as pellets. In case of fenofibrate, the compound was mixed stepwise with the powdered diet, followed by mixing with 2% agar and freeze-drying as pellets. Experimental diets were prepared freshly prior to start of the animal experiment and were stored at −20° C. (in darkness) during the experimental period. The composition of the high fat diet used is 24% casein, 20.4% dextrose, 24% fat, 18.67% maize flour and 6% cellulose.

Experimental design. Fifty six (n=8/group) male heterozygous APOE*3Leiden mice were transferred from the breeding facility to the experimental facility and fed a chow maintenance diet for 1 week to adapt to the new environment. At day 0, the mice were randomized on the basis of plasma lipid/triglyceride level into seven groups of eight mice each. All seven groups were then fed with the high fat diet as specified above. The animals were treated by gavage at a fixed time point, 5 consecutive days a week (at 16:00 pm on Monday to Friday, not on Saturday and Sunday; gavage volume: 150 μl) with the following solutions and according to the following scheme:

The control groups 1 and 4 also received the vehicle by gavage.

1. vehicle control (saline)

2. Lactobacillus rhamnosus GG in vehicle

3. Propionibacterium freudenreichii ssp. shermanii JS in vehicle

4. vehicle control (saline)

5. Lactobacillus rhamnosus GG in vehicle

6. Propionibacterium freudenreichii ssp. shermanii JS in vehicle

The dose of Lactobacillus rhamnosus GG or Propionibacterium freudenreichii ssp. shermanii JS was 10⁹ cfu/day. Groups 1, 2 and 3 received water ad lib. and groups 4, 5, and 6 received fat-free low-lactose milk ad lib. The milk was refreshed daily.

Positive control group (Group 7) was fed the high fat diet, with 0.003% (w/W) fenofibrate mixed into the diet.

TABLE 1
Experimental schedule

| Day of experi- | | |
|---|---|---|
| mental period | Action | Analysis |
| −7 to 0 | Adaptation period on chow | |
| 0 | Tail blood sample | Cholesterol (individually) |
| | Body weight | Triglycerides (individually) |
| | Randomization (based on | ALAT (group wise) |
| | plasma lipid/triglyceride) | SAA (individually) |
| | Start treatment | Fibrinogen (individually) |
| | | E-selectin or VCAM-1 |
| | | (individually) |
| | | Adiponectin (individually) |
| 7 | Body weight | |
| | Food intake | |
| 14 | Tail blood sample | Cholesterol (individually) |
| | Body weight | Triglycerides (individually) |
| | Food intake | ALAT (group wise) |
| | | SAA (individually) |
| | | Fibrinogen (individually) |
| | | E-selectin or VCAM-1 |
| | | (individually) |
| | | Adiponectin (individually) |
| 21 | Body weight | |
| | Food intake | |
| 28 | Tail blood sample | Cholesterol (individually) |
| | Body weight | Triglycerides (individually) |
| | Food intake | ALAT (group wise) |
| | Collection of the following | Lipoprotein profiles (group |
| | tissues: liver, gonadal and | level) by ÅKTA procedure |
| | visceral adipose tissue, | SAA (individually) |
| | muscle, prostate, ceocum, | Fibrinogen (individually) |
| | intestine | E-selectin or VCAM-1 |
| | | (individually) |
| | | Adiponectin (individually) |

EDTA plasma (tail blood, no anaesthesia) was obtained after a four-hour fast. Tail blood samples were taken between 12:00 and 13:00 on Tuesdays. To that end, a small incision was made in the tail vein using a scalpel and blood was collected directly in an EDTA-coated capillary tube.

Animals were sacrificed (CO/CO₂ mixture) at day 28 to collect tissues, i.e. liver, gonadal and visceral adipose tissue, muscle, ceocum, intestine (additionally performed) and prostate.

Measurements and Analytics

Total plasma cholesterol (kit Chol R1, Roche Diagnostics, Switzerland) in all mice individually.

Total triglycerides (kit Triglycerides GPO-PAP, Roche Diagnostics, Switzerland) in all mice individually.

ALAT (spectrophotometric assay, Reflotron system, Boehringer Mannheim) in pooled samples.

Lipoprotein distribution (ÅKTA procedure) in pooled samples (VLDL, IDL/LDL, HDL separation) (Verschuren L, et al., Arterioscler Thromb Vasc Biol. 2005; 25:161-167).

Lipid and Lipoprotein Analysis: Total plasma cholesterol and triglyceride levels were measured after 4-hour fasting using kits 1489437 (Roche Diagnostics) and 337-B (Sigma Aldrich Chemie BV), respectively. Lipoprotein profiles were obtained by using the AKTA-fast protein liquid chromatography system (Amersham Pharmacia) as described previously.

Plasma SAA (serum amyloid A protein; mouse SAA ELISA kit, Biosource, Belgium) and fibrinogen (in-house mouse fibrinogen ELISA) in all mice individually.

Soluble mouse E-selectin or mouse VCAM-1 (kits from R&D Systems) in all mice individually.

Evaluation of the effect of probiotic (Lactobacillus rhamnosus GG and Propionibacterium freudenreichii ssp. shermanii JS) for improving general health markers, and reducing plasma inflammation markers (as assessed by measuring SAA, fibrinogen, E-selectin, VCAM-1 in plasma) when applied to ApoE3Leiden mice alone or in combination with milk are shown in following examples 2-5.

### Example 2

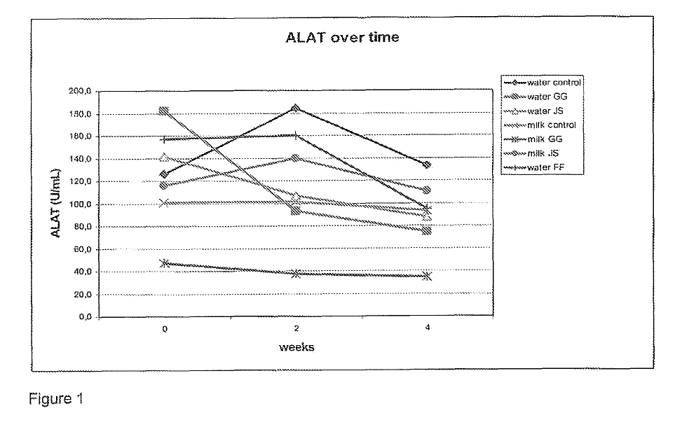

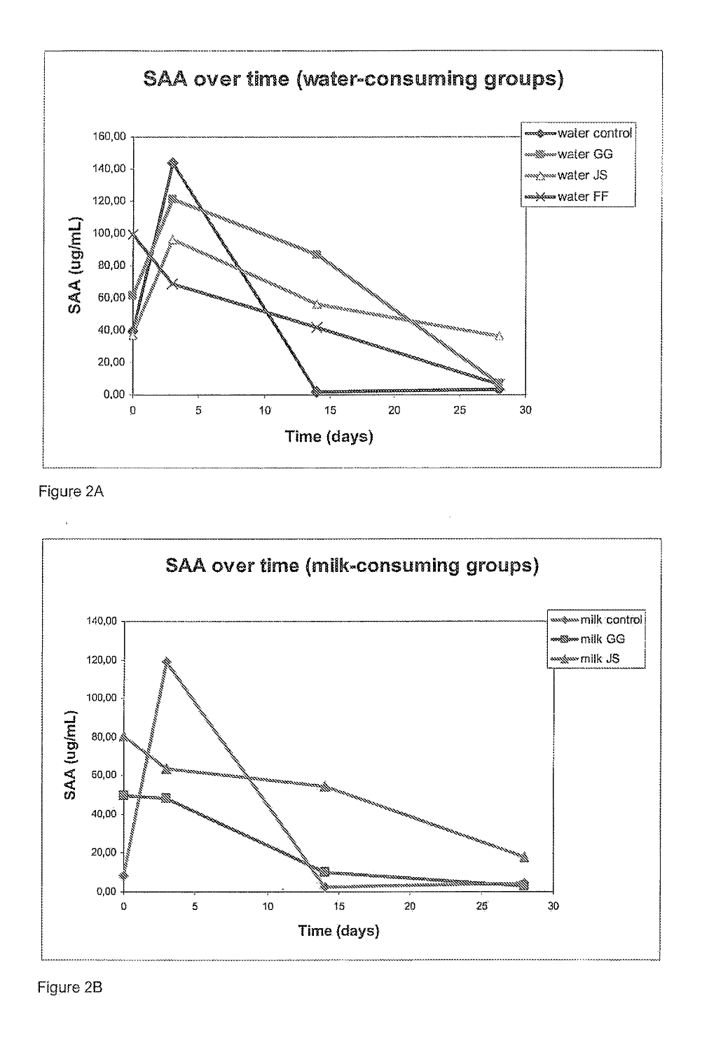

Effect of probiotic(s) on liver damage or activation was monitored by analysing alanine amino transferase (ALAT) activity. ALAT values as a measure for liver damage or liver activation were determined at group level and are presented as mean values. There were considerable differences between groups with respect to the initial baseline ALAT values, with group ALAT values between 182 U/mL (in water GG) and 34 U/mL (in milk GG) (FIG. 1). Since animals were matched into groups on basis of their baseline plasma cholesterol and triglyceride levels, group differences in baseline ALAT as observed here are possible.

In the water control group, the average plasma ALAT levels increased from 125 U/mL to 185 U/mL (t=2 weeks) and returned to the initial value at the end of the study (t=4 weeks). In the milk control group, plasma ALAT remained constant over time (about 100 U/mL), and in the positive control group (FF), ALAT remained constant during the first 2 weeks (about 160 U/mL) and subsequently decreased significantly at the t=4 w time point (95 U/mL; P<0.05 versus t=0).

A strong decrease in plasma ALAT values was observed already at 2 weeks in water-consuming probiotic groups. The decrease further continued resulting in even lower ALAT levels at t=4 w. To assess whether this ALAT-reducing effect became significant at t=4 w, we (additionally) determined individual ALAT levels for the groups at t=0 and t=4 w allowing to perform paired statistics (one-sided). In presence of water, probiotic significantly reduced ALAT levels as compared to their baseline (P<0.05). Also compared to the water control, ALAT was reduced significantly (P=0.038; 1-sided) in the water GG group.

Plasma ALAT values were significantly reduced compared to t=0 in the milk GG group. No effect on plasma ALAT was observed in the milk JS group.

Both probiotic markedly lowered plasma ALAT suggesting an improvement of liver functioning with no adverse effects on liver functioning at the concentrations used.

### Example 3

Effect of probiotic(s) on liver-derived inflammation markers was monitored by analyzing plasma SAA levels and fibrinogen. Plasma SAA and fibrinogen were detected in all mice individually as markers of the general inflammatory state. SAA is a type I (i.e. IL-1-inducible) acute phase protein whereas fibrinogen is a type II (i.e. IL-6-dependent) acute phase protein.

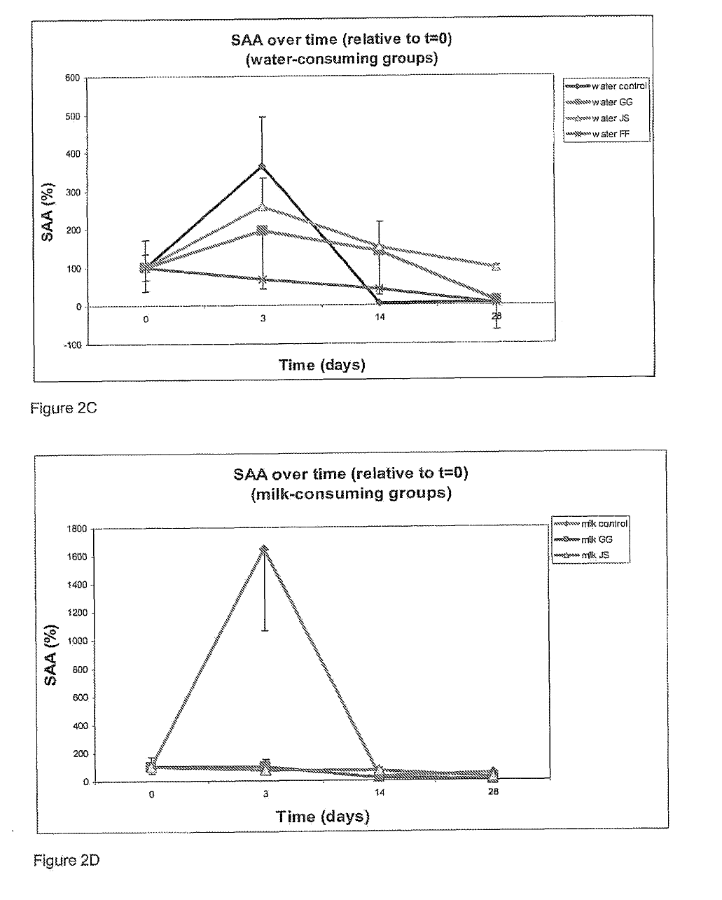

Water-consuming groups: High fat feeding had an acute effect on plasma SAA levels and increased plasma SAA significantly in the water control group (FIG. 2A) at 3 days, after which levels far below baseline with very low levels at 14 days and 28 days were obtained. Surprisingly, treatment with probiotic led to a less pronounced peak at 3 days together with a subsequent decrease to baseline level (JS) or far below baseline level (LGG), but in both cases at a much slower pace (more gradual) than seen with water control group. In positive control group suppression of SAA started from t=0 onwards (no peak at t=3 d) to reach low levels at t=28 d.

Grosso modo, the data indicate that the probiotics resemble positive control (fenofibrate) in suppressing high fat feed-induced acute inflammation at 3 days together with subsequent slow (gradual) decline in SAA levels also very similar to fenofibrate.

Milk-consuming groups: High fat feeding diet had an acute effect on plasma SAA levels and increased plasma SAA significantly at 3 days also in the milk control group (FIG. 2B), which demonstrated an acute inflammatory response in liver similar to water control group (FIG. 2A).

In the milk probiotic groups, induction of SAA expression by high fat diet feeding was fully suppressed and no peak was found at 3 days. Prolonged treatment with probiotic resulted in SAA levels that were lower than initial baseline levels. Together, this indicates that the probiotic exert anti-inflammatory effects resulting in reduced levels of the systemic inflammation marker SAA.

Relative SAA values: To correct for the differences in SAA at t=0, effects on SAA were also analyzed relative to the t=0 value, i.e. the t=0 value was set 100% for each group.

In the water control group, the increase in plasma SAA at 3 days relative to t=0 was significant (peak) (FIG. 2C). In the probiotic groups, the increase was not significant and the positive control group had significantly lower SAA values than the water control group. Similarly as in case of the water control group, SAA in the probiotic groups subsequently decreased to baseline level (JS) or far below baseline level (LGG), but in both cases at a much slower pace than in the water control group.

High fat feeding resulted in a pronounced and significant induction of SAA at 3 days in the milk control group (FIG. 2D). In both probiotic groups, the inflammatory response to high fat feeding was significantly quenched. At the end of the treatment period, the SAA levels of the probiotic groups were lower (significant in case of LGG) than in the milk control.

### Example 4

The effect of probiotic(s) on vascular inflammatory state was monitored by analysing E-selectin as a marker of the vascular inflammatory state. E-selectin is synthesized in endothelial cells and expression regulated by (=downstream of) IL-1 and TNF-α.

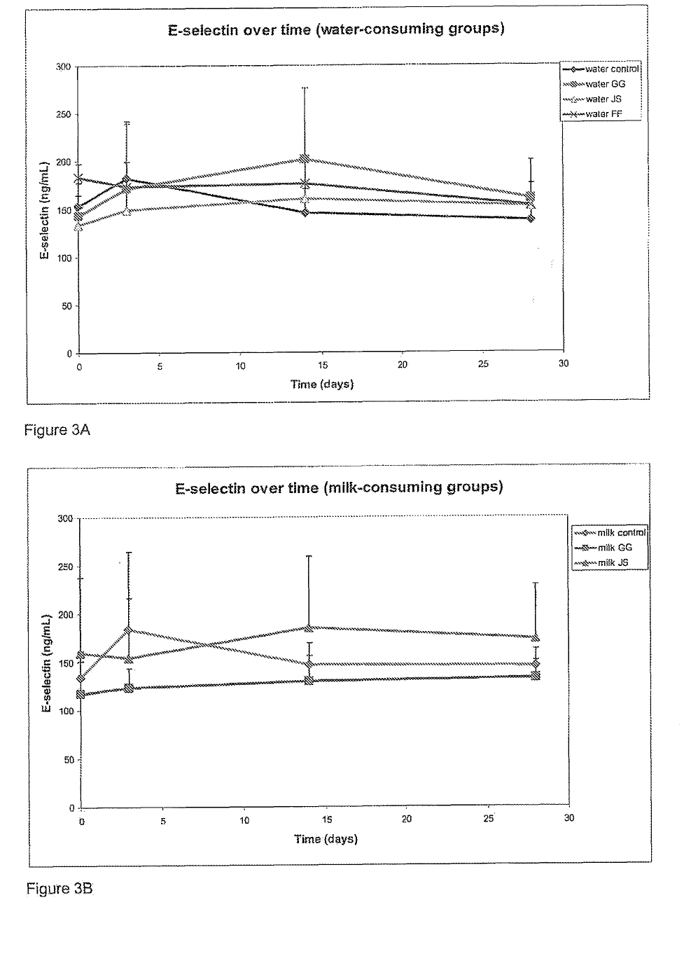

Water-consuming groups: Plasma E-selectin concentrations increased significantly in response to high fat feeding in the water control group, from 153 at t=0 to 183 ng/mL at 3 d and returned to levels that were comparable to baseline values at 14 days and 28 days (FIG. 3A). For the probiotic groups, the peak was obtained at 14 days. At 28 days plasma levels of E-selectin were still slightly elevated compared to baseline values in both probiotic group.

The high fat diet with 0.0035% fenofibrate induced no expression of E-selectin and plasma levels slightly decreased over time.

Milk-consuming groups: In the milk control group, plasma E-selectin concentrations increased significantly from 133 to 183 ng/mL at 3 days (FIG. 3B). E-selectin levels remained slightly, but significantly elevated at 14 days and 28 days, when compared to baseline values. Probiotic suppressed the high fat diet-induced expression of E-selectin at 3 days. Prolonged treatment with probiotic did not reduce E-selectin levels below baseline levels (cf. positive control).

Together, these data indicate that probiotic treatment can suppress acute vascular inflammation elicited by high fat diet feeding, similar as for liver-derived SAA.

### Example 5

The effect of probiotic(s) on vascular inflammatory state was monitored by analysing VCAM-1 as a marker of the vascular inflammatory state. It is also synthesized in endothelial cells and expression regulated by (=downstream of) IL-1 and TNF-α.

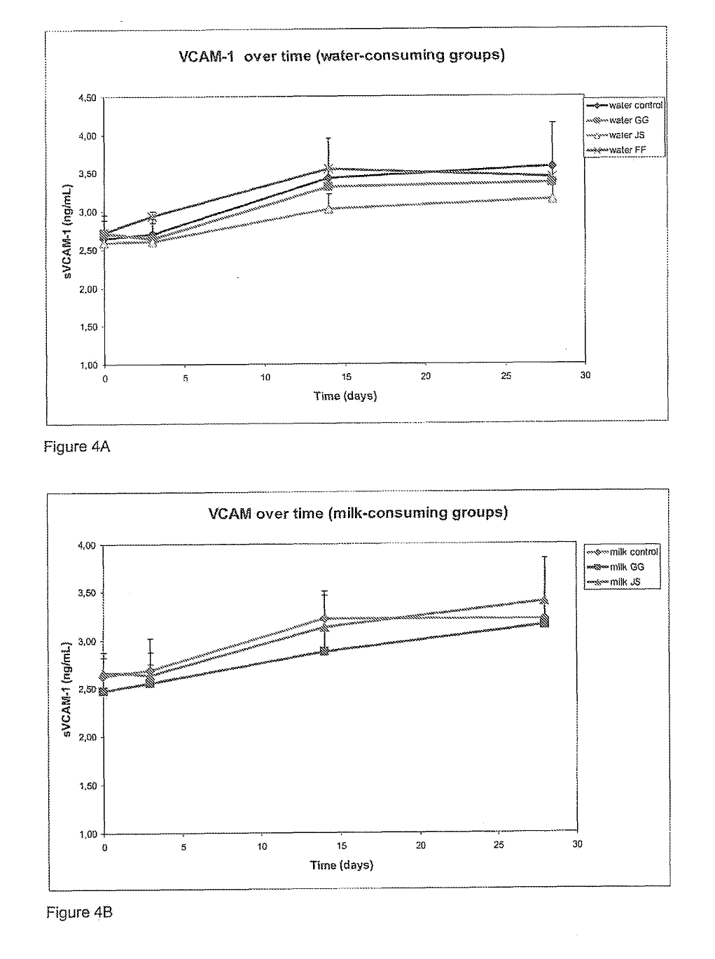

Water-consuming groups: The increases in VCAM-1 expression in the water control, probiotic groups and positive control group were comparable, with the increase least pronounced in the JS group (FIG. 4A). Anti-inflammatory effect of JS on VCAM-1 expression was statistically significant at 14 days and 28 days.

Milk-consuming groups: Similarly, VCAM-1 levels increased over time in response to high fat diet feeding, with significant increase for all milk-consuming groups at 14 days and at 28 days (FIG. 4B). Both probiotic groups showed a similar increase in VCAM-1 expression relative to baseline as the milk control group, indicating absence of an effect of probiotics on VCAM-1 in presence of milk.

### Example 6

To monitor the effect of probiotic(s) on general animal health during the experimental period, food intake, body weight, and liver weight were determined.

Food intake was reduced in all milk-consuming groups, most probably as a result of the extra caloric intake through milk. There were no apparent effects of the probiotic per se on food intake. Also, there were no major effects on body weight, except for the water JS group which displayed a significantly lower body weight and the milk LGG group which displayed a significantly higher body weight at the end of the study period.

Liver weight was lower in the probiotic treated groups when compared to the control groups; the livers of which were somewhat heavier than untreated chow-fed ApoE3Leiden control animals suggesting that high fat feeding increased liver weight. The increase normally seen in liver weight in response to high fat feeding (ultimately leading to liver steatosis) was less pronounced in the probiotic treated groups, with JS being more potent. Together, this may be suggestive for a protective effect of the probiotic on liver functioning and hepatic fat accumulation.

### Example 7

Effect of probiotic(s) on plasma lipids was monitored. The probiotic had no effect on total plasma cholesterol and total plasma triglyceride (TG) levels, except in the presence of milk when both LGG and JS reduced plasma TG. A more refined analysis of lipoproteins by fractionation of lipoproteins in atherogenic VLDL and LDL; and atheroprotective HDL showed absence of major effects of the probiotic in the presence of water, but, importantly, showed an improvement of the plasma lipoprotein profile in the presence of milk (mainly by LGG): LGG reduced atherogenic IDL/LDL and increased beneficial HDL with respect to both the cholesterol and the phospholipids content of the respective lipoprotein fractions. The effects of LGG in combination with milk were more pronounced than the effects of fenofibrate (positive control).

### Example 8

Effect of probiotic(s) on inflammation markers was monitored. The probiotics had no major effect on the IL-6-/STAT3-/CEBPβ-controlled inflammation marker as deduced from fibrinogen (a type II acute phase protein) and adiponectin analysis, suggesting that the anti-inflammatory effect seen with the probiotics is not a general, but more restricted (i.e. mainly affecting IL-1-/NF-κB-regulated genes). In this respect, the probiotics differ from fenofibrate, which reportedly exerts more global anti-inflammatory effects, also involving fibrinogen.

### Example 9

Effect of probiotic(s) on adipose tissue content was monitored. Treatment with JS significantly reduced the gonadal adipose tissue mass in the presence of water. In the presence of milk, an insignificant decrease of gonadal and visceral adipose tissue was found. The plasma adiponectin levels of the JS groups were lower than LGG and the respective control groups.

1-30. (canceled)

31. A method of alleviating, preventing and/or treating diet-induced low-grade inflammation comprising administering to an individual in need of such treatment an effective amount of a probiotic.

32. A method of alleviating, preventing and/or treating a disorder and a disease related to and/or associated with diet-induced low-grade inflammation comprising administering to an individual in need of such treatment an effective amount of a probiotic.

33. A method of normalizing an abnormal diet-induced low-grade inflammation marker comprising administering to an individual in need of such treatment an effective amount of a probiotic.

34. A method of suppressing a diet-induced low-grade inflammation marker comprising administering to an individual in need of such treatment an effective amount of a probiotic.

35. The method according to claim 33, wherein the marker is formed in the liver, adipose tissue and/or vasculature.

36. The method according to claim 31, wherein, the low-grade-inflammation is caused by fats in the diet or by high-fat and/or hyperlipidemic diet.

37. The method according to claim 31, wherein the probiotic is selected from the group consisting of lactobacilli, bifidobacteria, propionibacteria, lactococci, enterococci, streptococci, and yeast, and any combinations thereof.

38. The method according to claim 37, wherein the probiotic is Lactobacillus rhamnosus LGG (ATCC 53103), Lactobacillus rhamnosus LC705 (DSM 7061), and/or Propionibacterium freudenreichii ssp. shermanii JS (DSM 7067) or a mixture thereof.

39. The method according to claim 31, wherein the probiotic is combined with a prebiotic.

40. The method according to claim 31, wherein the probiotic(s) is formulated into an edible or an enterally or an orally administered product.

41. The method of claim 40, wherein the product is a product of the dairy industry, beverage industry, or pharmaceutical industry, or it is a natural product.

42. The method of claim 41 wherein the product is a dairy product, drink, juice, soup or children's food.

43. A method of decreasing the risk of developing metabolic syndrome, obesity, cardiovascular diseases and/or diabetes type 2, comprising administering to an individual in need of such treatment an effective amount of Lactobacillus rhamnosus LGG (ATCC 53103), Lactobacillus rhamnosus LC705 (DSM 7061) and/or Propionibacterium freudenreichii ssp. shermanii JS.

44. A method of preventing, alleviating and/or treating metabolic syndrome, obesity, cardiovascular diseases and/or diabetes type 2, comprising administering to an individual in need of such treatment an effective amount of Lactobacillus rhamnosus LGG (ATCC 53103), Lactobacillus rhamnosus LC705 (DSM 7061) and/or Propionibacterium freudenreichii ssp. shermanii JS.
